(19) United States
(12) Patent Application Publication
(10) Pub. No.: US 2008/0154109 A1
(43) Pub. Date: Jun. 26, 2008

(54) Pelvic Catheter for Cervical Measurement

(76) Inventor: Bolanle Sogade, Macon, GA (US)

Correspondence Address: MICHAEL I KROLL, 171 STILLWELL LANE, SYOSSET, NY, 11791, US

(21) Appl. No.: 11/615,624

(22) Filed: Dec. 22, 2006

(51) Int. Cl. A61B 5/0444

(52) U.S. Cl. 600/376; 600/478; 600/562

(57) ABSTRACT

The present invention relates to a catheter and a control unit for providing visual images, measurements, and for performing gynecological procedures. The catheter has a tip with a light and at least one sensor. The catheter is connected to the control unit and the control unit converts the signal from the catheter into measurements or visual images. The information may be displayed on a LCD screen or printed on paper to provide the user with a hard copy. The catheter may include a blade, an electrode, or a pressure inducing or relieving source.

### BACKGROUND OF THE INVENTION

1. Field of the Invention

The present invention relates generally to catheters and, more specifically, to an intra vaginal catheter having an elongated tubular construction for introduction into a patient for intervention of labor or birth. The device is utilized for primarily reading and imaging the cervix where the catheter tip has a light source and imaging source. The catheter tip may be provided with inter-constructed or attached electronic sensors, an extendible blade, or tissue sample mechanism. The present invention employs a control unit for providing a visual display of the images or data collected by the catheter and/or providing a hard copy printout. The present invention provided useful information pertaining to a labor or birth, while also presenting a mechanism for recording health related data or functions in any other application of a gynecological procedure.

2. Description of the Prior Art

There are other catheter devices designed for gynecological applications. While said catheters may be suitable for the purposes for which they were designed, they would not be as suitable for the purposes of the present invention, as hereinafter described.

### SUMMARY OF THE PRESENT INVENTION

A primary object of the present invention is to provide an intra vaginal catheter used for providing light for reading and imaging a patient's cervix during an intervention pertaining to labor or delivery.

Another object of the present invention is to provide an intra vaginal catheter utilized for obtaining and graphing cervical health related data of a patient.

Yet another object of the present invention is to provide an intra vaginal catheter having electronic sensors built into or attached to its tip for the monitoring and graphing of data relating to a pregnancy or cervical health.

Still yet another object of the present invention is to provide an intra vaginal catheter having an extendible and retractable blade for the artificial rupturing of internal structures relating to a pregnancy.

Another object of the present invention is to provide an intra vaginal catheter having pressure-delivering capabilities for the intervention of a pregnancy.

Yet another object of the present invention is to provide an intra vaginal catheter having a control unit for gathering and graphing of obtained data.

Additional objects of the present invention will appear as the description proceeds.

The present invention overcomes the shortcomings of the prior art by providing an intra vaginal catheter having an illuminating source and imaging components along with a blade, electronic sensors, and an electrode for the monitoring of a cervix or intervention of a pregnancy. Additionally the present invention has a control unit having data recording and printing means. The present invention may be provided with the ability to deliver pressure or suction at the catheter tip and/or with a mechanism to extract tissue samples for other gynecological procedures.

The foregoing and other objects and advantages will appear from the description to follow. In the description reference is made to the accompanying drawing, which forms a part hereof, and in which is shown by way of illustration specific embodiments in which the invention may be practiced. These embodiments will be described in sufficient detail to enable those skilled in the art to practice the invention, and it is to be understood that other embodiments may be utilized and that structural changes may be made without departing from the scope of the invention. In the accompanying drawing, like reference characters designate the same or similar parts throughout the several views.

The following detailed description is, therefore, not to be taken in a limiting sense, and the scope of the present invention is best defined by the appended claims.

### BRIEF DESCRIPTION OF THE DRAWING FIGURES

In order that the invention may be more fully understood, it will now be described, by way of example, with reference to the accompanying drawing in which:

FIG. 1 is a perspective view of the apparatus of the present invention.

FIG. 2 is a detailed view of the light tip probe of the present invention.

FIG. 3 is a detailed view of the light tip probe of the present invention

FIG. 4 is a function chart of the apparatus of the present invention.

FIG. 5 is an illustrative view of the present invention.

FIG. 6 is an illustrative view of the present invention.

FIG. 7 is an illustrative view of the present invention.

FIG. 8 is an illustrative view of the present invention.

FIG. 9 is an illustrative view of the present invention.

FIG. 10 is a detailed view of the light tip probe of the present invention.

### LIST OF REFERENCE NUMERALS

With regard to reference numerals used, the following numbering is used throughout the drawings.

10 Present Invention

12 Catheter

14 Tip

16 Connector

18 Light Source

20 Image Means

22 Integrated Sensor

24 Attached Sensor

26 Blade

28 Tissue Sample Means

30 Pressure Means

32 Electrode

34 Control Unit

36 Liquid Crystal Display

38 Print Out

40 Print Out Port

42 Vagina

44 Cervix

46 Uterus

48 Fetus

50 Amniotic Sac

52 Uterine Wall

54 LightBeam

56 Image Recording Device

58 Calibration Device

### DETAILED DESCRIPTION OF THE PREFERRED EMBODIMENT

The following discussion describes in detail one embodiment of the invention (and several variations of that embodiment). This discussion should not be construed, however, as limiting the invention to those particular embodiments, practitioners skilled in the art will recognize numerous other embodiments as well. For definition of the complete scope of the invention, the reader is directed to appended claims.

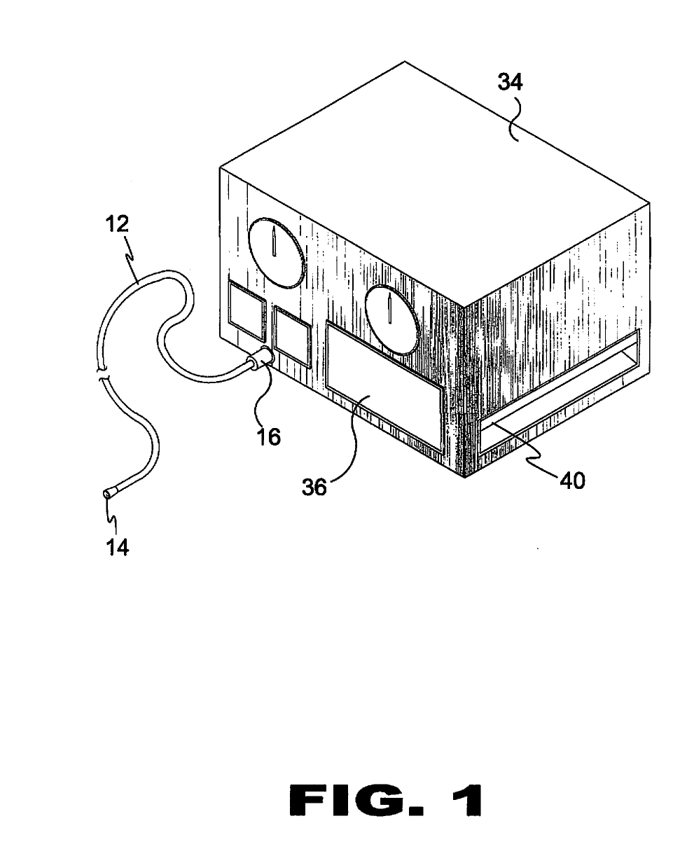

FIG. 1 is a perspective view of the apparatus of the present invention 10. The present invention 10 is catheter 12 attached to an image recording apparatus or control unit 34 for providing images and measurements of the cervix 44. The images or measurements may be displayed on a screen or a hard copy may be printed for archival purposes. The information received by the control unit 34 from the catheter 12 may be transcribed into chart form. The tip 14 of the catheter 12 will have a light source 18 and image means 20. The light source 18 provides adequate light to enable the image means 20 to provide the control unit 34 with a visual image of what the image means 20 “sees”. The image means may be a camera or other image-gathering device. The catheter 12 may have other elements attached thereon or integrated therein. These additional elements may be an integrated sensor 24, an attached sensor 24, a blade 26, tissue sample means 28, pressure means 30, or an electrode 32. The previous elements are given as examples and are not meant to limit the scope of the present invention. The catheter 12 of the preset invention may incorporate any combination of additional elements if more than one additional element is required to perform a specific procedure or if it is desired to make the catheter 12 more versatile by enabling the device to perform a variety of procedures. The additional elements allow the catheter 12 to be utilized in a variety of procedures such as but not limited to taking tissue samples, rupturing the membrane(s) of the patient (typically the amniotic sac 44), measuring pressure inside the amniotic sac 44, performing amnioinfusion, or measuring fetal heart rate.

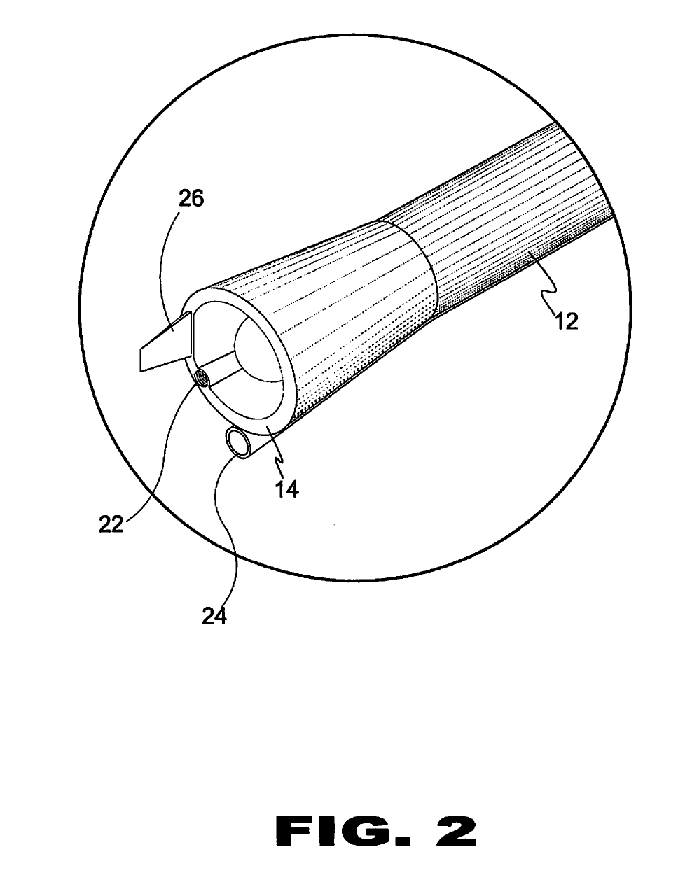

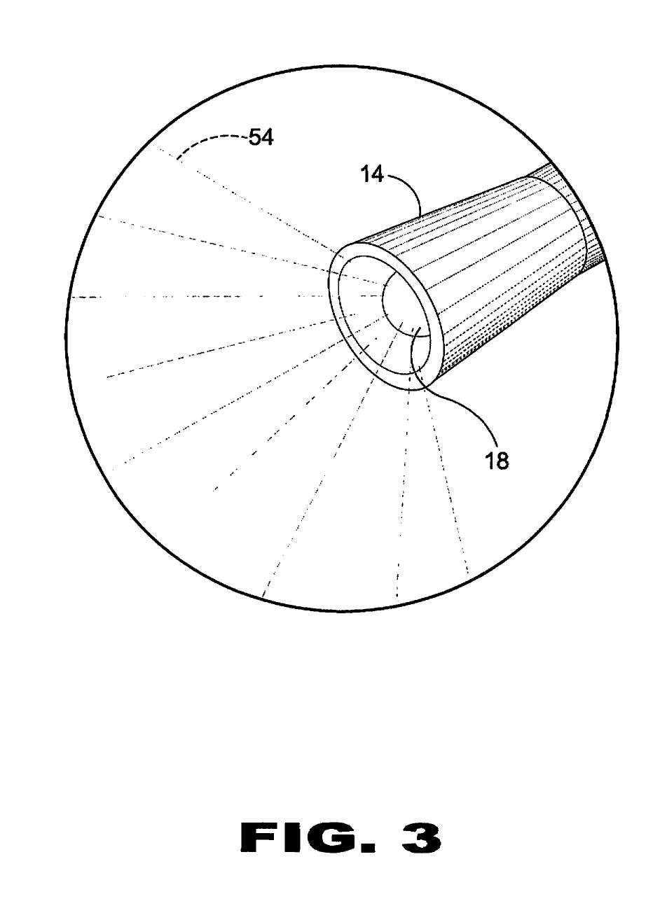

A detailed view of the end or tip 14 of the catheter 12 of the present invention 10 is seen in FIG. 2. The tip 14 of the catheter 12 is preferably semi-rigid allowing its introduction into the patient through the vagina 42. It is envisioned that the tip 14 may be made more or less rigid depending upon the use envisioned for the catheter 12. The tip 14 of the catheter 12 includes a light source 18 to provide light that is necessary for visual imaging and may also include other elements as noted previously. The sensor(s) (22,24) provide the control unit with the necessary data to provide precise measurements. The tip 14 may include a blade 26. It is envisioned that the blade 26 is retractable and extendable. This allows the positioning of the catheter 12 prior to the blade 26 being exposed. This enables the user to install the catheter 12 without causing damage to the patient. When the catheter 12 is in position then the user extends the blade 26 so that the desired task may be performed. Once the task has been accomplished the user retracts the blade 26 to allow removal of the catheter 12. The catheter 12 has a tubular body with is protected by flexible and insulated covering. The covering encloses and protects the internal components of the catheter 12. The catheter 12 has a connector 16 for joining the catheter 12 the control unit 34. It is envisioned that the catheter 12 of the present invention may be employed on pregnant and non-pregnant patients. FIG. 3 is an illustration showing the light emitting from the light source 18. It is envisioned that the pressure means 30 will allow the user to induce pressure or flow to the tip of the catheter, or to provide suction at the tip of the catheter. It is also envisioned that a sensor may be provided to the user with the pressure inside the amniotic sac. It is also envisioned that the tip 14 of the catheter 12 be provided with a tissue sample means 28. The tissue sample means 28 may simply be an extendible probe that is capable of grasping or securing a piece of tissue. The tissue sample means 28 may be an extendible and retractable hollow needle. The tissue sample means 28 may be any other suitable mechanism that is capable of obtaining a tissue sample.

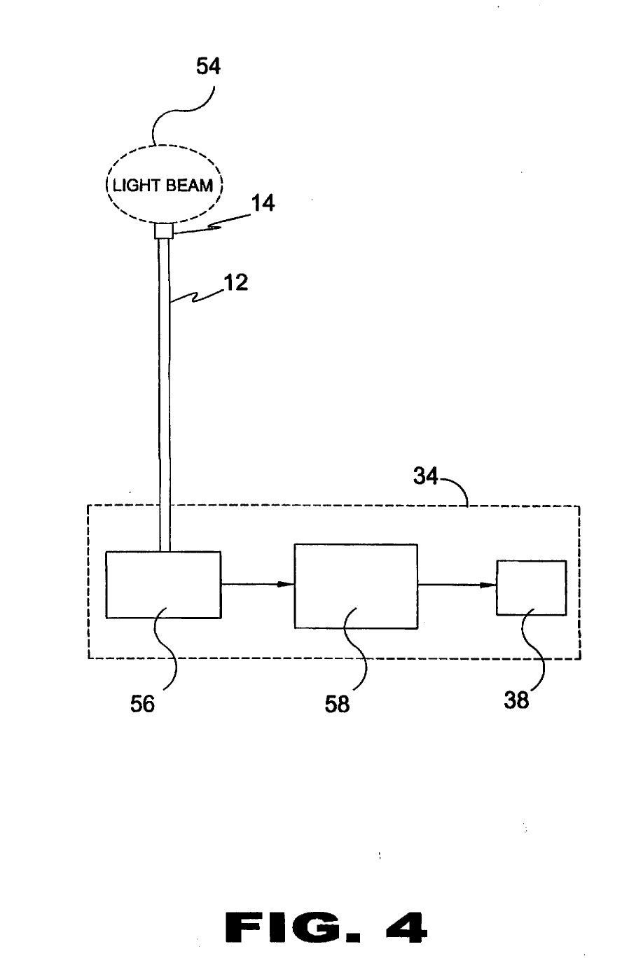

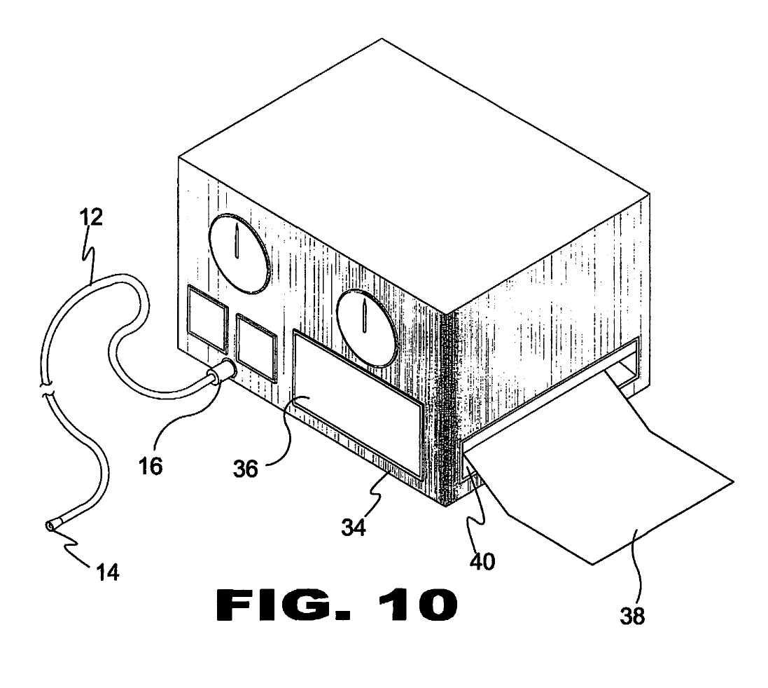

The control unit 34 of the present invention 10 is seen in FIGS. 1 and 10. The control unit 34 records and calibrates the signal(s) received from the catheter so that they can seen on a visual display such as monitor or so that they may be printed out in hard copy form. The monitor may be a Liquid Crystal Display (LCD) 36 or any other suitable monitor. The control unit 34 interprets the signals provided by the catheter 12 sensors in order to provide measurements, pressure readings, fetal heart rate, or other desired information. FIG. 4 shows a function chart of the present invention 10. The control unit 34 may employ an image-recording device 56 for recording the images or signals received from the catheter 12. The image-recording device 56 may be a hard drive for storing and retrieving large amounts of data. It is also envisioned that the control unit 34 be provided with the capability of recording data on removable media such as but not limited to compact disks (CDs), digital video disks (DVDs), memory chips, flash drives, or any other suitable removable media. It is envisioned that a drive may be provided for reading and writing on CD and DVD formatted disks. At least one USB port may be provided with for connection to a flash or jump drive. The flash or jump drives allow the user to quickly transport data recorded therein record to another computer or other device. The control unit 34 will provide the user with visual images that will be displayed on the LCD 36. The signals received from the catheter 12 are interpreted or calibrated to provide accurate measurements or other reading such as but not limited to fetal heart rate or amniotic pressure.

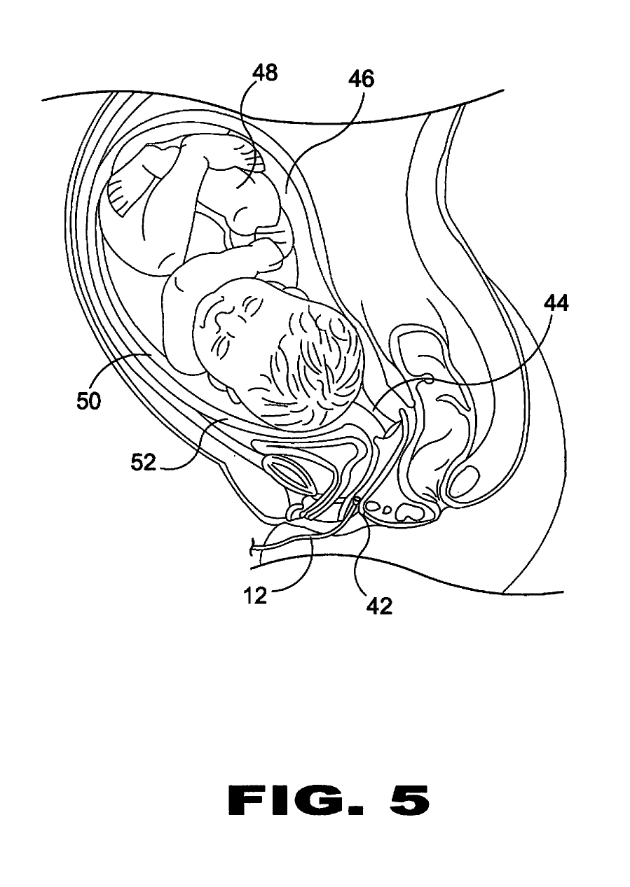

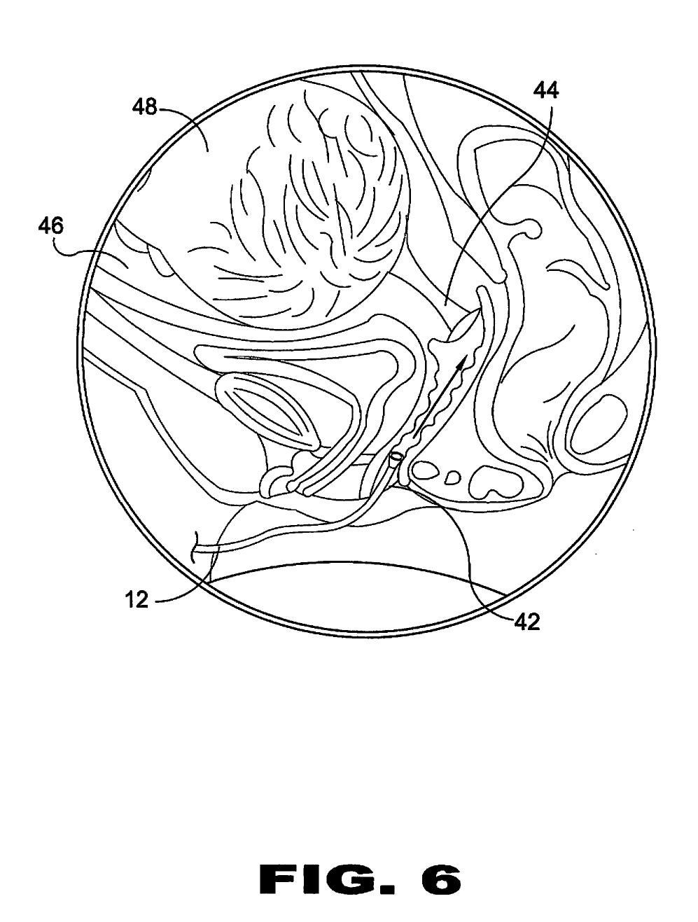

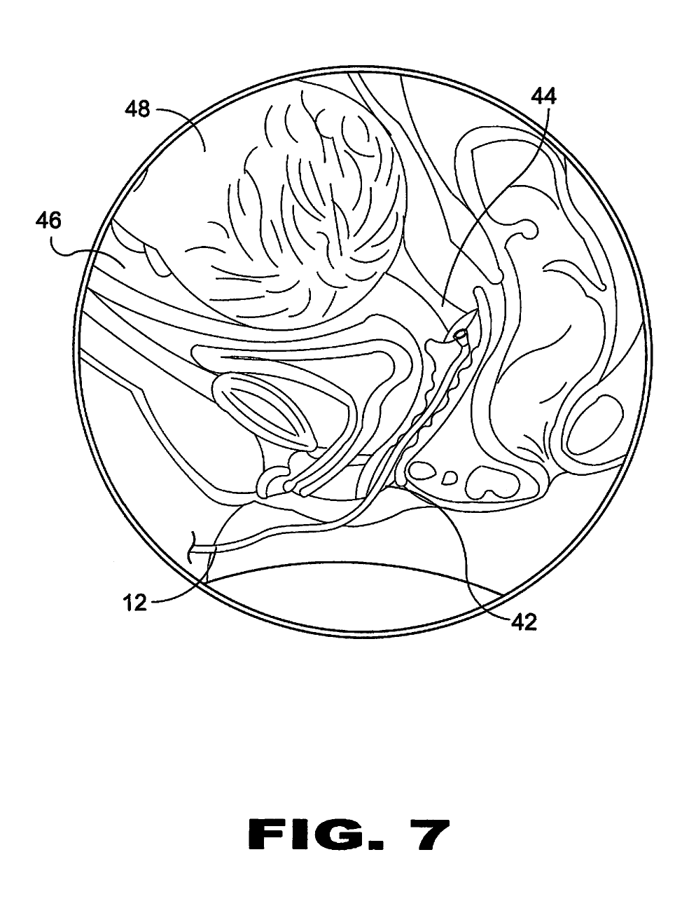

FIGS. 5-7 show the present invention 10 in use on a pregnant patient. The catheter 12 is introduced into the vagina 42 of the patient. The tip 14 of the catheter 12 is snaked into and through the vagina 42 and then into the cervix 44. The catheter measures and provides visual images the cervix 44. FIG. 7 shows the catheter 12 about to enter the cervix 44. The catheter 12 may be positioned in the cervix 44 for recording images and measurements therein. As the cervix dilates the measurements are recorded and displayed by the control unit 34 to provide the user with current measurements without performing a physical examination of the cervix 44. It is also envisioned that the catheter 12 may be positioned in the vagina 42 for continual measurement of the entrance to the cervix 44.

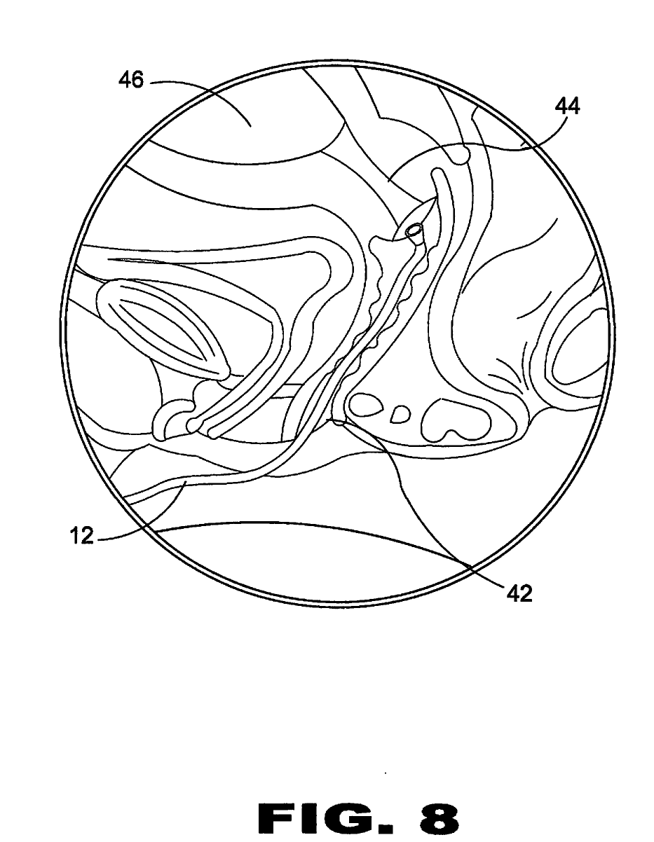

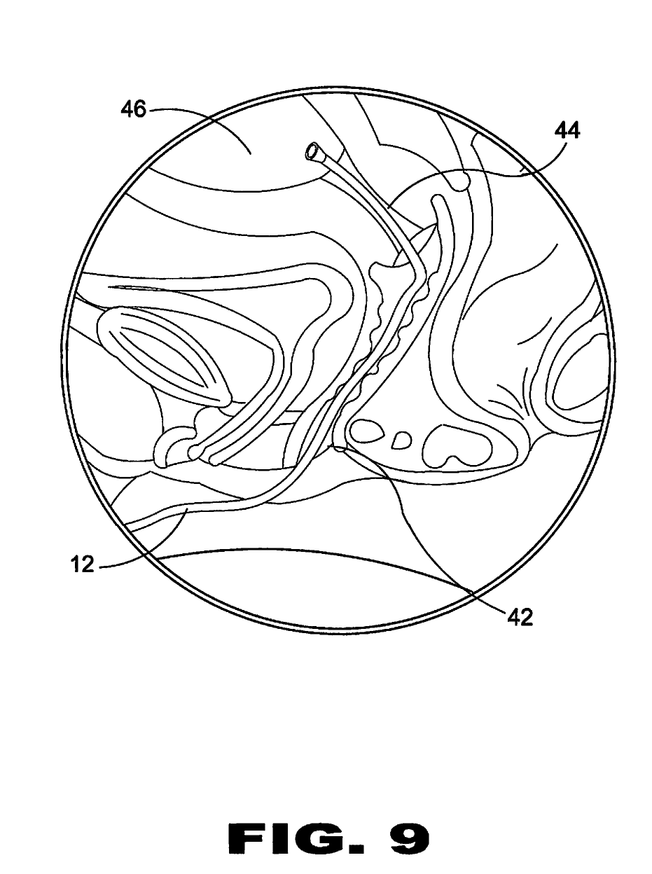

FIGS. 8 and 9 show the present invention 10 in use on a patient that is not pregnant. The catheter 12 is snaked through the vagina 42, cervix 44 and into the uterus 46 for intrauterine procedures. The catheter 12 may be employed for vaginal, cervical, or uterine procedures. As previously noted the catheter 12 may be provided with tissue sample means 28 to remove and return tissue samples. The catheter 12 may be employed to provide visual images of the vagina 42, the cervix 44 or uterus 46.

I claim:

1. A catheter for introduction into a patient, the catheter comprising:
a flexible insulated member having a distal end and a proximal end, the proximal end having a connector attached thereon, the distal end terminating in a semi rigid tip, the tip having a light and at least one sensor thereon, wherein the light and the sensor are capable of providing a signal through the catheter to the connector whereby the signal can be interpreted into a measurement or a visual image.

2. The catheter of claim 1, wherein the tip has a retractable and extendible blade disposed thereon.

3. The catheter of claim 1, wherein the sensor is integrated into the tip of the catheter and does not extend beyond any exterior surface of the tip.

4. The catheter of claim 1, wherein the sensor is attached to an exterior surface of the tip.

5. The catheter of claim 2, wherein the tip includes pressure means for inducing or relieving pressure at the tip.

6. The catheter of claim 1, wherein the tip includes an electrode capable of measuring the heart rate of a fetus.

7. The catheter of claim 2, wherein the tip includes an electrode capable of measuring the heart rate of a fetus.

8. The catheter of claim 1, wherein the tip includes means for retrieving a tissue sample.

9. A cervical measuring device comprising:
a flexible insulated catheter having a distal end and a proximal end, the proximal end having a connector attached thereon, the distal end terminating in a semi rigid tip, the tip having a light and at least one sensor thereon, wherein the light and the sensor are capable of providing a signal through the catheter to the connector whereby the signal can be interpreted into a measurement or a visual image; and
a control unit connected to the catheter wherein a signal received from the catheter is recorded by the control unit and is converted into discrete measurements.

10. The device of claim 9, wherein the tip of the catheter has a retractable and extendible blade disposed thereon.

11. The device of claim 9, wherein the sensor is integrated into the tip of the catheter and does not extend beyond any exterior surface of the tip.

12. The device of claim 9, wherein the sensor is attached to an exterior surface of the tip.

13. The catheter of claim 10, wherein the tip includes pressure means for inducing or relieving pressure at the tip.

14. The catheter of claim 9, wherein the tip includes an electrode capable of measuring the heart rate of a fetus.

15. The catheter of claim 10, wherein the tip includes an electrode capable of measuring the heart rate of a fetus.

16. The catheter of claim 9, wherein the tip includes means for retrieving a tissue sample.

17. A system for performing gynecological procedures, the system comprising:
a flexible insulated catheter having a distal end and a proximal end, the proximal end having a connector attached thereon, the distal end terminating in a semi rigid tip, the tip having a light and at least one sensor thereon, wherein the light and the sensor are capable of providing a signal through the catheter to the connector whereby the signal can be interpreted into a measurement or a visual image; and
a control unit connected to the catheter wherein a signal received from the catheter is recorded by the control unit and is converted into discrete measurements.

18. The device of claim 17, wherein the tip of the catheter has a retractable and extendible blade disposed thereon.

19. The device of claim 17, wherein the sensor is integrated into the tip of the catheter and does not extend beyond any exterior surface of the tip.

20. The device of claim 17, wherein the sensor is attached to an exterior surface of the tip.

21. The catheter of claim 18, wherein the tip includes pressure means for inducing or relieving pressure at the tip.

22. The catheter of claim 17, wherein the tip includes an electrode capable of measuring the heart rate of a fetus.

23. The catheter of claim 18, wherein the tip includes an electrode capable of measuring the heart rate of a fetus.

24. The catheter of claim 17, wherein the tip includes means for retrieving a tissue sample.
